(19) United States
(12) Patent Application Publication
(10) Pub. No.: US 2021/0052161 A1
(43) Pub. Date: Feb. 25, 2021

(54) FLUORESCENCE IMAGING SYSTEM

(71) Applicant: Rebound Therapeutics Corporation, Irvine, CA (US)

(72) Inventors: Ross Tsukashima, Irvine, CA (US); Milo Alfaro Amorsolo, Irvine, CA (US)

(73) Assignee: Rebound Therapeutics Corporation, Irvine, CA (US)

(21) Appl. No.: 16/550,159

(22) Filed: Aug. 23, 2019

(51) Int. Cl. A61B 5/00; A61K 49/00; A61B 17/34

(52) U.S. Cl. CPC A61B 5/0071; A61B 5/7425; A61B 5/0077; A61B 2017/3425; A61K 49/0034; A61B 17/3423; A61K 49/0036

(57) ABSTRACT

A system for fluorescence guided surgery which includes alternatingly obtaining white light images and blue light images and simultaneously displaying those images on a display screen, such that a surgeon is presented with immediate, real-time video which shows the target tissue under white light and fluorescing tissue under blue light on the same screen, at the same time. The images may be presented side-by-side, or superimposed.

### FIELD OF THE INVENTIONS

The inventions described below relate to the field of minimally invasive surgery for the treatment of tumors.

### BACKGROUND OF THE INVENTIONS

Fluorescence Guided Surgery is a technique used to identify cancerous tumors and cancerous cells during surgery. Under broad spectrum light (white light), there is no clear difference in the appearance of cancerous tumor and cancerous cells and surrounding healthy tissue, especially at the margins of the cancerous tumor. Especially in the brain, a surgeon wants to remove cancerous tissue while avoiding disruption of healthy tissue, but the difficulty in discerning one from the other makes this difficult. Improved visualization of the cancer can help ensure that all cancerous tissue has been removed while reducing damage to healthy tissue such as nerves, blood vessels, and brain tissue.

To use the Fluorescence Guided Surgery technique, a fluorescent agent is administered to the patient. Shortly after administration, the fluorescent agent is absorbed by cancerous tissue, but not by healthy tissue surrounding the cancerous tissue. A surgeon may use white light while exploring a surgical workspace created to gain access to the cancerous tissue, and while manipulating surgical tools to excise the cancerous tissue, and intermittently use narrow spectrum light to cause the fluorescent agent in the cancerous tissue to fluoresce to make it possible to identify cancerous tissue and delineate tumor margins. However, during narrow spectrum illumination, the surgical workspace is dark, being illuminated with only the excitation light, so that surrounding healthy tissue cannot be clearly seen. In this technique, the surgeon must repeatedly switch back and forth between white light and excitation light, find the cancerous tissue under excitation light, and switch back to white light while excising the cancerous tissue to ensure that the surgeon avoids removing healthy tissue. Switching back and forth between light sources may require manual replacement of filters on a light source.

For glioma (a type of tumor in the brain), 5-ALA (5-Aminolevulinic acid) is a preferred agent for inducing fluorescence in the glioma. 5-ALA-induced tumor fluorescence occurs because 5-ALA is taken up by malignant glioma cells and metabolized within glioma cells into the fluorescent metabolite, protoporphyrin IX (PpIX). 5-ALA is preferred for visualizing malignant brain tumors and surrounding infiltrating cancer cells outside of the tumor because it preferentially accumulates in the cancer cells. 5-ALA is metabolized into the fluorescent compound protoporphyrin-IX (PpIX). Thus, though 5-ALA is not itself fluorescent, it is pro-fluorescent in the sense that it metabolizes into a compound that is fluorescent. When protoporphyrin IX (PpIX) is illuminated with blue light, it glows red, so that cancerous tissue containing protoporphyrin IX (PpIX) stands out clearly from surrounding brain tissue under blue light. This is called “5-aminolevulinic acid (ALA)-induced protoporphyrin IX (PpIX) fluorescence.” For glioblastoma (another type of cancerous tumor in the brain), heptamethine dye (heptamethine carbocyanine, for example), which is fluorescent, is a suitable agent for inducing fluorescence. Heptamethine carbocyanine fluoresces under near-infrared light. Thus, the narrow spectrum excitation light differs, depending on the fluorescent agent used in fluorescence guided surgery.

### SUMMARY

The devices and methods described below provide for improved visualization of diseased tissue within the body of a patient during minimally invasive surgery. The device includes a surgical access port, a camera positioned to view a body tissue within a surgical workspace through the surgical access port, a broad spectrum light source, an excitation spectrum light source, and a control system operable to (1) operate the light sources to illuminate target tissue within the workspace with both the broad spectrum light source and an excitation spectrum light source which may cause fluorescence of compounds in diseased tissue and (2) generate video images for presentation to a surgeon on a display in a manner which assists in visualizing both the target tissue under the broad spectrum light and any fluorescing diseased tissue under the excitation light. For example, where blue light is used in conjunction with 5-ALA, the system alternatingly obtains white light images and blue light images and simultaneously displays those images on a display screen, such that a surgeon is presented with immediate, real-time video which shows the target tissue under white light and red fluorescing tissue under blue light on the same screen, at the same time. The images may be presented side-by-side, or superimposed.

The method entails placement of the camera and lights and a suitable support structure, if needed (a surgical access port, for example) proximate a surgical workspace which may include diseased tissue. The surgeon will operate the camera and its control system to obtain images of the target tissue and diseased tissue. The control system is operable to obtain video images through the camera, obtaining, in rapidly alternating fashion, video images of the target tissue under broad spectrum light and frames of the target tissue under narrow spectrum excitation light, and generating corresponding video images for presentation on a display screen. (1) The control system may be configured to operate the display screen to present, in rapidly alternating fashion, video images of the target tissue under broad spectrum light (typically, white light) and frames of the target tissue under narrow spectrum excitation light (blue light, for example), in the same position in the display screen, so that narrow spectrum excitation light images are superimposed on broad spectrum light images, preferably alternating so rapidly that the surgeon may not perceive flickering between the two images. (2) The control system may be configured to operate the display screen to present, simultaneously, side-by-side video images of the target tissue under narrow spectrum excitation light and frames of the target tissue under narrow spectrum excitation light. The alternating illumination light source is accomplished rapidly, through operation of the control system (energizing and de-energizing the light sources), rather than through repeated operator input into the control system, so that the surgeon is free to continue manipulation of tools in the workspace without interruptions necessary to switch between views, and continue, for example, to excise, ablate, macerate and aspirate diseased tissue visible under blue light, while avoiding disruption of healthy tissue which cannot clearly be seen under blue light, without having to switch to white light to ensure that the tools are not disrupting healthy tissue.

The method may entail administering a fluorescence-inducing agent to the patient. In this case, the imaging method described above will be performed after administration of the fluorescence-inducing agent and its uptake by diseased tissue. The fluorescence-inducing agent may be any agent which may be administered to a patient to induce fluorescence in diseased tissue of interest. Fluorescence-inducing agents are preferentially absorbed or attached to diseased tissue on or within target tissue in the workspace and may include (1) a fluorescence agent capable of fluorescence when illuminated with an excitation light source, or, (2) in the case of 5-ALA and other compounds, a fluorescence pro-agent which itself may or may not be fluorescent but is metabolized in the body into a fluorescence agent, either before or after absorption into the diseased tissue (3) a fluorescence aggregator capable of attaching to an endogenous fluorescence agent (one naturally occurring in the body) and thereafter preferentially depositing in the diseased tissue or (4) in the case of reduced nicotinamide adenine dinucleotide (NADH), an endogenous fluorescence agent that is naturally occurring within diseased tissue at a higher density than healthy surrounding tissue.

The fluorescence-inducing agent may be administered through any route, including oral administration (5-ALA), injection into the blood stream, injection into the target tissue, or splashing onto target tissue. After allowing sufficient time for the fluorescence-inducing agent to be taken up by diseased tissue within or on the target tissue, a surgeon will illuminate the target tissue with broad spectrum light as necessary to see the target tissue and manipulate tools to work on the target tissue, and illuminate the target tissue with narrow spectrum excitation light to see diseased tissue within or on the target tissue and manipulate tools within the target tissue.

In one mode of operation, the control system is configured to correlate blue light images with the energization of the blue light source, and white light images with energization of the white light source, to generate the side-by-side display with images in appropriately corresponding sections of the display, to visualize cancerous tissue in the brain, where the cancerous tissue is expected to have taken up previously-administered 5-ALA. In other modes of operation, the control system may be configures to correlate infrared light images with energization of an infrared light source and white light images with energization of the white light source, to generate the side-by-side display with images in appropriately corresponding sections of the display, to visualize cancerous tissue in the brain, where the cancerous tissue is expected to have taken up previously-administered indocyanine green (ICG).

### BRIEF DESCRIPTION OF THE DRAWINGS

FIG. 1 illustrates a patient with a blood mass in the brain that necessitates surgical intervention, with a cannula which has been inserted into the brain, with the distal end of the cannula proximate the blood mass and an obturator tip extending into the blood mass.

FIGS. 2 and 3 illustrate a cannula system useful for implementation of the method of visualizing diseased tissue with fluorescence imaging.

FIGS. 4 and 5 are video images of screen displays that may be provided by the cannula system.

FIGS. 6 and 7 are video images of screen displays that may be provided by the cannula system.

FIG. 8 is a flow chart representing the operation of the control system to generate video images for interleaved display on the display screen.

### DETAILED DESCRIPTION OF THE INVENTIONS

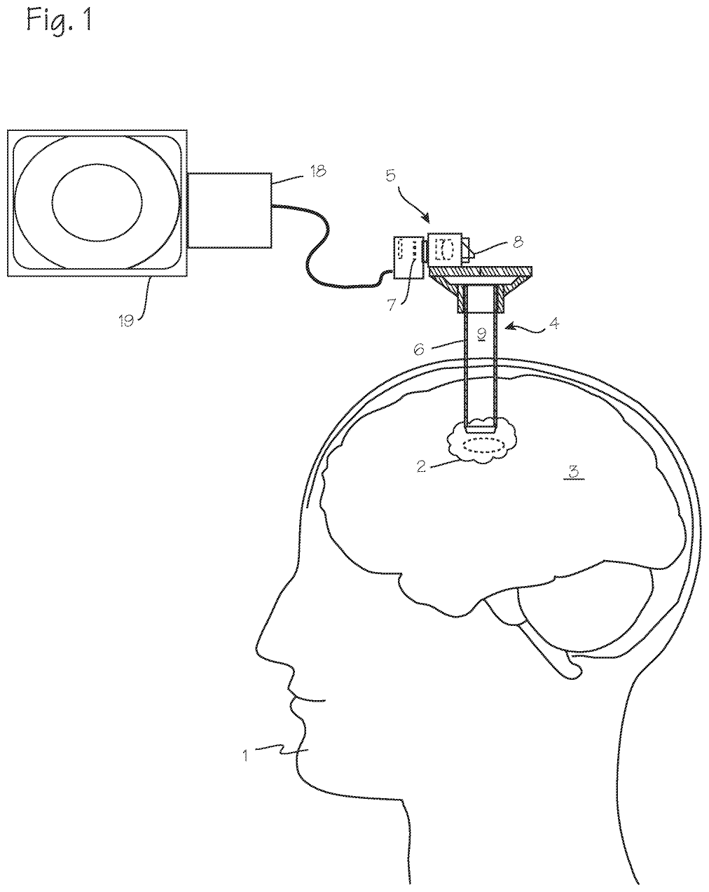

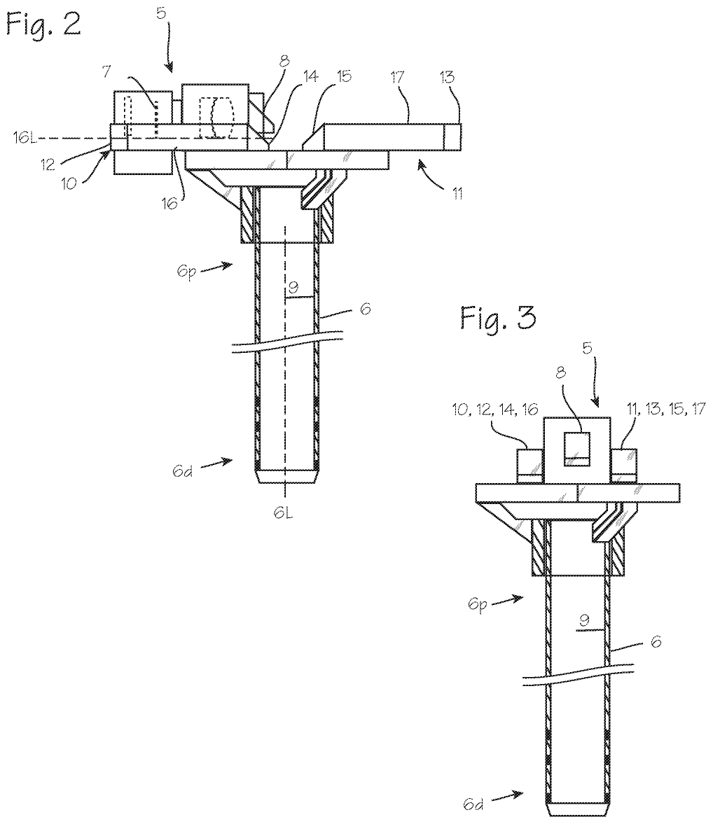

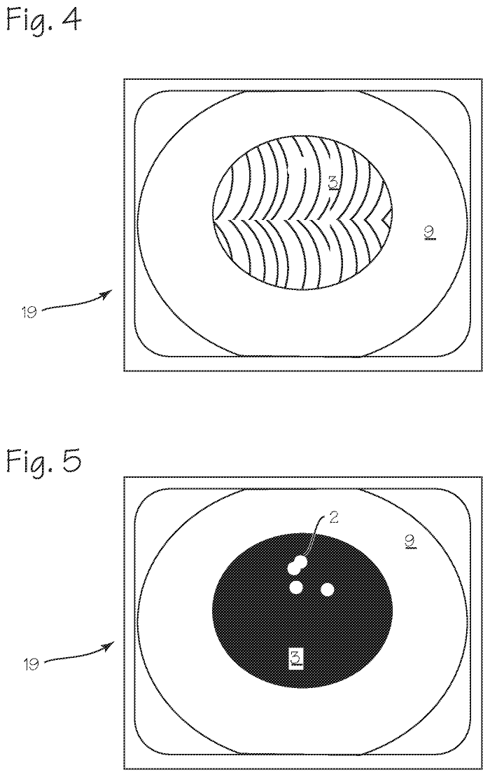

FIGS. 1, 2 and 3 illustrate a cannula system that may be conveniently used to implement the imaging method describe in relation to FIGS. 4 through 8 in a minimally invasive surgery. FIG. 1 illustrates a patient 1 with diseased tissue 2 in the brain 3 that necessitates surgical intervention, with a cannula 4 which has been inserted into diseased tissue, with the distal end of the cannula proximate the diseased tissue. The diseased tissue may be a glioma or glioblastoma in the brain, an ependymoma in the spine, or other diseased tissue. A camera 5 is mounted on the proximal rim of the cannula, with a portion of the camera overhanging the rim of the cannula and disposed over the lumen of the cannula, and is operable to obtain video or still images of the distal end of the cannula lumen, including target tissue at the distal end of the cannula such as the brain and any diseased tissue in the brain. As shown in both FIGS. 1, 2 and 3, the cannula comprises a cannula tube 6 with the camera assembly 5 secured to the proximal end 6p of the cannula, and with a distal end 6d adapted for insertion into the body of the patient. The camera assembly includes an imaging sensor 7 and a prism, reflector or other mirror structure or optical element 8, overhanging the lumen 9 of the cannula tube. Preferably, for use in the brain, a portion of the camera assembly, such as the prism, reflector or mirror, extends into the cylindrical space defined by the lumen of the cannula tube and extending proximally beyond the proximal end of the cannula, and is spaced from the proximal end of the cannula, and extends only slightly into the cylindrical space. As shown in FIGS. 2 and 3, the cannula also includes lighting assemblies 10 and 11 which include light sources 12 and 13 and associated optics, if any, which in the illustrated embodiment include prisms 14 and 15, and lenses 16 and 17, which may be used in this configuration to direct light from light sources into the lumen and toward target tissue. FIG. 1 also shows the control system 18, which is configured and operable to operate the light sources, obtain video image data captured by the camera, and generate/translate corresponding video image data for display on the display screen 19.

One light source is operable to provide a broad spectrum light useful for generally illuminating the target tissue, and the other light source is operable to provide high intensity narrow spectrum excitation light for illuminating any fluorescence agent in the target tissue. The broad spectrum light may be white light of any preferable color temperature. The narrow spectrum excitation light is provided in a color which causes the fluorescence agent to fluoresce, and this depends on the particular fluorescence agent. For example, if the fluorescence-inducing agent is 5-ALA, the excitation light should be blue (380-440 nm (visible blue light)) to cause emission of red light (620-634 nm (visible red light)) depending on the environment, and if the fluorescence-inducing agent is heptamethine dye, the excitation light should be near-infra-red (775 nm and 796 nm), to cause emission of infrared light (808 nm and 827 nm), and if the fluorescence-inducing agent is ICG, the excitation light should be red, to cause emission of infrared light, depending on the environment. The light sources are preferably LED's or other small light sources that can readily be disposed on the proximal end of the cannula tube, but other light sources may be used, such as lasers or remote light boxes coupled with fiber optics or waveguides, and the light sources may be disposed on the distal end of the cannula tube. When used in conjunction with the cannula, each lighting assembly and each light source is configured to illuminate the target tissue through the cannula.

Other fluorescence-inducing agents may be used in the method, including fluorescein (460-500 nm blue/green light to emit 510-530 nm green light, depending on surrounding tissue); Methylene blue (MB) (670 nm red light results in emission of 690 nm red/near infrared light); and indocyanine green (ICG) (red or near-infrared light at 750 to 800 nm results in emission of infrared light, and wavelengths over 800 nm depending on the surrounding tissue).

FIGS. 4 and 5 are images of the target tissue that may be provided by the cannula system on a display screen 19. FIG. 4 is a screen shot illustrating the display, as generated by the control system, of the target tissue (the brain, in this illustration) obtained while the target tissue 3 is illuminated with white light from the broad spectrum lighting assembly 10. The image includes the inner wall of the cannula tube 6. In this image, diseased tissue, if any, in the field of view cannot be clearly discerned (any fluorescence is swamped by the bright broad spectrum light). FIG. 5 is a screen shot illustrating the display, as generated by the control system, of the target tissue obtained while the target tissue is illuminated with narrow spectrum excitation light from the narrow spectrum excitation lighting assembly 11. In this image, assuming 5-ALA is used as the fluorescence-inducing agent, diseased tissue 2 is clearly visible because PpIX within the diseased tissue is glowing bright red, but healthy target tissue in the field of view cannot be clearly discerned. The healthy tissue may be visible in the image to the extent that it reflects the NES light, which for 5-ALA would be blue.

In a basic mode of operation, the surgeon would operate the cannula system to illuminate the target tissue with white light to obtain an image of the target tissue, and then illuminate the target tissue with blue light to obtain an image of any diseased tissue, and operate resection and or aspiration tools, inserted through the cannula tube, to remove the diseased tissue, manually operating an interface to switch back and forth between white light illumination and blue light illumination, as necessary for the surgeon to see the diseased tissue and manipulate the tools to remove the diseased tissue.

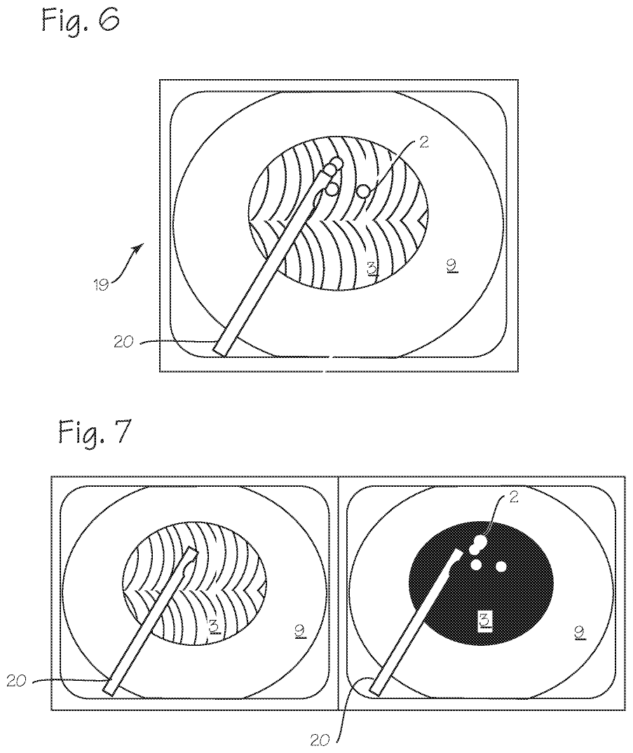

FIGS. 6 and 7 are alternative images of the target tissue that may be provided by the cannula system on a display screen 19. In FIG. 6, the control system has been operated to obtain images of the target tissue 3 under white light, and obtain images of the target tissue and diseased tissue 2 under blue light (again, the narrow spectrum excitation light color may differ, depending on the fluorescence-inducing agent use to induce fluorescence), and generate an image for display on the display screen which includes images of the diseased tissue superimposed on images of the target tissue. In FIG. 7, the control system has been operated to generate displayed images of the target tissue under white light, and under blue light, and the control system has operated the display to present both images side-by-side.

To obtain and display these video images, the control system is operable to obtain video data from the camera assembly, processing the video data, and presenting corresponding displayed video images on the display screen. To provide smooth, real-time video images of both the target tissue and the fluorescent diseased tissue on the display, in synchronous fashion (that is, the images in white light and the images in blue light are presented simultaneously, so that the surgeon can view, simultaneously, video images of the white light field and the blue light field), the control system is configured to alternately (1) operate the white light source to illuminate the target tissue with white light and obtain one or more video frames of the target tissue (2) operate the blue light source to illuminate the target tissue with blue light and obtain one or more video frames of the target tissue and any diseased tissue, and (3) simultaneously present images of the target tissue obtained under white light and images of the target tissue obtained under blue light, where the control system accomplishes the alternating illumination/imaging at a rapid rate. Where the fluorescence-inducing agent is expected to induce fluorescence with emission of non-visible wavelengths (infrared, near-infrared, or ultraviolet), the camera assembly will include sensors sensitive to the non-visible wavelengths, and the control system will be configured to process captured video images to color-shift the images of fluorescing diseased tissue into a visible color for display in the displayed video images. Where the fluorescence-inducing agent is expected to induce fluorescence with emission of visible wavelengths (red), which may be confused with blood, the control system may be configured to process captured video images to color-shift the images of fluorescing diseased tissue into any preferred color for display in the displayed video images.

The images may be displayed side-by-side, as shown in 7, where video of white light images are shown in one section of the display screen and blue light images are shown in a second section of the display screen. Because the images are obtained simultaneously, the surgeon will see any movement of tool tips or tissue in both images. Thus, the surgeon need not manually or otherwise volitionally switch between views to see diseased tissue and healthy target tissue. Each motion of the tool tip 20 appears simultaneously on both sections of the display, and each resection of diseased tissue is simultaneously visible on both sections (though resected tissue may not appear to be distinct from healthy tissue on the white light image.

To accomplish simultaneous side-by-side display with images obtained through the single camera assembly, the control system synchronizes illumination with broad spectrum light with capturing at least one frame of a video image of the target tissue while illuminated under broad spectrum light, and illumination with narrow spectrum excitation light (blue, for 5-ALA) with capture of at least one frame of a video image of the target tissue while illuminated under narrow spectrum excitation light, and then displays broad spectrum light images with narrow spectrum excitation light images simultaneously on the display screen.

The broad spectrum light images and narrow spectrum excitation light images may be presented on the display as shown in FIG. 6, in which the narrow spectrum excitation light images are superimposed on the broad spectrum light images. To provide this image, the control system may operate in a simple mode, in which all captured video images are transmitted, without correlating which is obtained under which source.

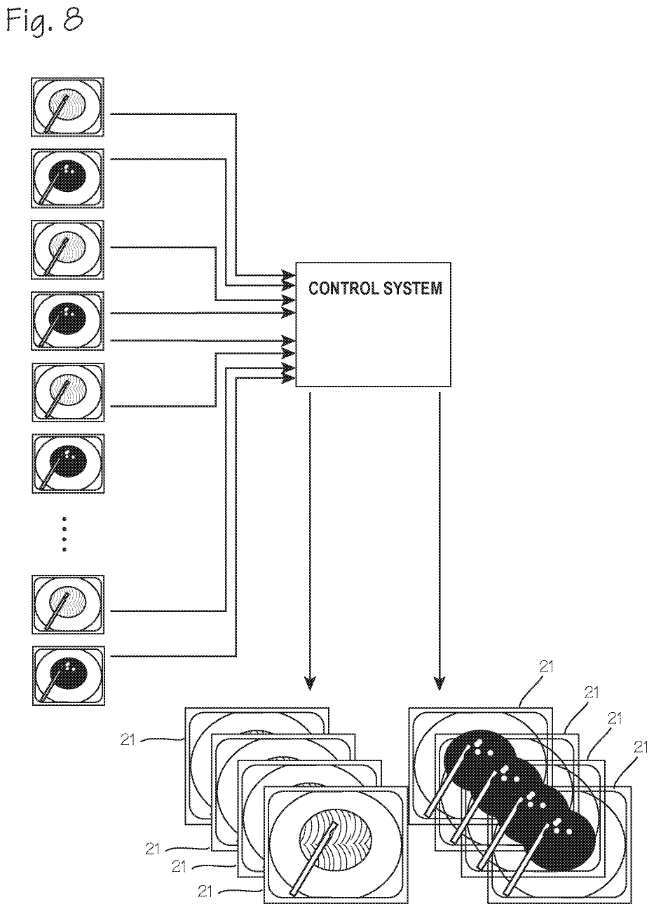

FIG. 8 is a flow chart representing the operation of the control system to generate video images for interleaved display on the display screen. The control system operates the camera, the broad spectrum light source and that excitation light source to obtain a first “frame” of a captured video image under broad spectrum light, then a second “frame” of a captured video image under excitation light, then a third “frame” of a captured video image under broad spectrum light, then a fourth “frame” of a captured video image under excitation light, and so on, including many frames under both broad spectrum and narrow spectrum excitation light. Images under broad spectrum light and images under excitation light are obtained from the same camera. The control system also tracks which frames are obtained under broad spectrum light, and which frames are obtained under excitation light, and uses the frames obtained under broad spectrum light to generate a displayed video image 21 of the target tissue obtained under broad spectrum light, and uses frames obtained under excitation light to generate a displayed video image 22 of the diseased tissue obtained under excitation light. In this mode of operation, the control system is operated to display the displayed video image 21 obtained under broad spectrum white and the displayed video image 22 obtained under excitation light at the same time, so that the surgeon can see both at the same time.

The frames of the target tissue obtained under broad spectrum light and frames of the target tissue obtained under narrow spectrum excitation light are preferably captured by the camera, and generated and delivered to the display screen as displayed video image frames at a frame rate sufficient to present smooth video, perceived by an observer of the display screen with minimal or no perception of flicker. Sufficiently fast frame rates currently used for video ranges from 12 frames per second and higher, movies are typically presented at 24 frames per second, and PAL, SECAM and NTSC and HDTV use various frame rates, as high a 60 frames per second. Twelve frames per second is considered the lowest frame rate that will result in the illusion of smooth movement. If smooth movement is desired, then, in the displayed images, the control system may operate to capture at least 12 frames per second under broad spectrum light, and at least 12 frames per second under narrow spectrum excitation light (interleaved, as described above, capturing one frame under broad spectrum light, then one frame under narrow spectrum excitation light, then one frame under broad spectrum light, then one frame under narrow spectrum excitation light, and so on). Thus using currently available video camera technology, operating the camera itself at 24 frames per second, the control system can be configured to obtain 12 frames per second under broad spectrum light, and at least 12 frames per second under narrow spectrum excitation light (interleaved one for one), and display side by side video of the broad spectrum illuminated images and narrow spectrum excitation illuminated images each at 12 frames per second. Although explained with the example of 1:1 interleaving (capturing one frame under broad spectrum light, then one frame under narrow spectrum excitation light, then one frame under broad spectrum light, then one frame under narrow spectrum excitation light, and so on) and corresponding 1:1 display of displayed frames on the display screen (displaying one frame obtained under broad spectrum light, then one frame obtained under narrow spectrum excitation light, then one frame obtained under broad spectrum light, then one frame obtained under narrow spectrum excitation light, and so on), the captured video frames may be captured at different rates under each light source, and displayed video frames may be displayed at different rates, so that the ratio of captured and/or displayed broad spectrum frames may differ from a strict 1 to 1 ratio. For example, narrow spectrum excitation illuminated frames may be obtained at a ratio of 1 for every 2 broad spectrum illuminated frames, and displayed at a similar ratio.

The control system comprises at least one processor and at least one memory including program code with the memory and computer program code configured with the processor to cause the system to perform the functions described throughout this specification. Software code may be provided in a software program in a non-transitory computer readable medium storing the program, which, when executed by a computer or the control system, makes the computer and/or the control system communicate with and/or control the various components of the system to accomplish the methods, or any steps of the methods, or any combination of the various methods, described above.

Multiple fluorescence inducing agents can be administered to the patient to support fluorescence guided surgery. For example, for resection of glioma in the brain, which might be located very near some small blood vessels which a surgeon would want to avoid damaging, two fluorescence inducing agents may be administered in order to induce different fluorescence distinct tissue. For example, 5-ALA may be administered to induce fluoresce of glioma (glowing red), while ICG may be administered to induce fluoresce in blood vessels (glowing infrared), and the target tissue can be illuminated with excitation light for both (blue for the 5-ALA, and near-infrared for ICG). After administration and during surgery, the target tissue can be illuminated by excitation light including excitation light for both agents, and the control system can be operated as above, to obtain images of the diseased tissue and blood vessels within the healthy tissue, so that the surgeon can see both on the display screen, and attack the diseased tissue while avoiding the now-visible underlying blood vessels. To accomplish this, the cannula system may be augmented with an additional excitation light source, matched to the additional agent, and the control system may be further configured to (1) illuminate the target tissue with a second excitation light source in a third period and obtain captured video data during the third period and present three side by side images, (2) illuminate the target tissue with a second excitation light source in a third period and obtain captured video data during the third period and a single image of the target area composed of the broad spectrum image, the second excitation light (blue) image, and the second excitation light (infrared) image or (3) a mixture of display modes, in which two of the images are presented in superimposed fashion in a composite image while the third is shown side-by-side with the composite image.

The imaging system and method are illustrated above in the context of a cannula system which provides a particularly useful platform for use of the imaging system in brain surgery and spinal surgery. The benefits of the imaging system and method may be achieved with or without the cannula, in other platforms, including open surgery with separately supported components, or in endoscopic surgery with lighting and imaging components provided one or more tools in an endoscopic workspace.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. An imaging system for use in fluorescence-guided surgery, said imaging system comprising:
a first camera assembly operable to obtain video images;
a broad spectrum light source configured to illuminate a target tissue within the body of a patient;
a narrow spectrum excitation light source configured to illuminate the target tissue with narrow spectrum light suitable for causing fluorescence in a fluorescence agent;
a control system configured to operate the camera, broad spectrum light source and narrow spectrum excitation light source, and an associated display screen; wherein
the control system is configured to alternatingly (1) operate the broad spectrum light source to illuminate the target tissue with broad spectrum light and obtain one or more video images of the target tissue from the first camera while the target tissue is illuminated with broad spectrum light and (2) operate the narrow spectrum excitation light source to illuminate the target tissue with narrow spectrum excitation light and obtain one or more video images of the target tissue and any diseased tissue from the first camera while the target tissue is illuminated with narrow spectrum excitation light, and wherein the control system accomplishes said steps of alternatingly illuminating and obtaining images at a frame rate sufficient to create displayed video images of both (1) the target tissue and any diseased tissue from the first camera while the target tissue is illuminated with broad spectrum light; (2) the target tissue and any diseased tissue from the first camera while the target tissue is illuminated with narrow spectrum excitation light;
the control system is further configured to operate the display screen to simultaneously present the video images of both (1) the target tissue and any diseased tissue from the first camera while the target tissue is illuminated with broad spectrum light and (2) the target tissue and any diseased tissue from the first camera while the target tissue is illuminated with narrow spectrum excitation light.

2. The imaging system of claim 1, wherein:
the control system is configured to present the video images of both (1) the target tissue and any diseased tissue from the first camera while the target tissue is illuminated with broad spectrum light; (2) the target tissue and any diseased tissue from the first camera while the target tissue is illuminated with narrow spectrum excitation light in different sections of a display.

3. The imaging system of claim 1, wherein:
the control system is configured to present the video images of both (1) the target tissue and any diseased tissue from the first camera while the target tissue is illuminated with broad spectrum light; (2) the target tissue and any diseased tissue from the first camera while the target tissue is illuminated with narrow spectrum excitation light in a single composite video image, with images of the target tissue and any diseased tissue from the first camera while the target tissue is illuminated with narrow spectrum excitation light superimposed on images of the target tissue and any diseased tissue from the first camera while the target tissue is illuminated with broad spectrum light.

4. The imaging system of claim 1, wherein:
the narrow spectrum excitation light configured to illuminate the target tissue with blue light suitable for causing fluorescence in protoporphyrin IX (PpIX).

5. The imaging system of claim 1, wherein:
the narrow spectrum excitation light is configured to illuminate the target tissue with near-infrared light suitable for causing fluorescence in indocyanine green (ICG).

6. The imaging system of claim 2, wherein:
the narrow spectrum excitation light configured to illuminate the target tissue with blue light suitable for causing fluorescence in protoporphyrin IX (PpIX).

7. The imaging system of claim 2, wherein:
the narrow spectrum excitation light configured to illuminate the target tissue with near-infrared light suitable for causing fluorescence in indocyanine green (ICG).

8. The imaging system of claim 3, wherein:
the narrow spectrum excitation light configured to illuminate the target tissue with blue light suitable for causing fluorescence in protoporphyrin IX (PpIX).

9. The imaging system of claim 3, wherein:
the narrow spectrum excitation light configured to illuminate the target tissue with near-infrared light suitable for causing fluorescence in indocyanine green (ICG).

10. The imaging system of claim 1, further comprising:
a cannula having a proximal end and a distal end, and a lumen extending from said proximal end to said distal end, said distal end adapted for insertion to the body of a patient; wherein
the camera, broad spectrum light source, narrow spectrum excitation light source are disposed on the proximal end of the cannula, and said camera is configured to obtain images of the target tissue, and the broad spectrum light source is configured to illuminate the target tissue through the cannula, and the narrow spectrum excitation light source is configured to illuminate the target tissue through the cannula.

11. A method of visualizing diseased tissue and healthy tissue in a surgical workspace, said method comprising the steps of:
administering a fluorescence-inducing agent to the patient; wherein said fluorescence-inducing agent is preferentially absorbed or attached to diseased tissue on or within target tissue in the workspace;
placing a first video camera in position to obtain video images of the target tissue;
placing a broad spectrum light in position to illuminate the target tissue;
placing a narrow spectrum excitation light in position to illuminate the target tissue, wherein said narrow spectrum excitation light source has a wavelength which will induce fluorescence in a fluorescing agent corresponding to the fluorescence-inducing agents;
operating a control system to alternatingly operate the broad spectrum light and the narrow spectrum excitation light to illuminate the target tissue;
operating the first video camera, through the control system, to obtain video images through the first camera, to obtain, in rapidly alternating fashion, video images of the target tissue under white light and video images of the target tissue under blue light; and
operating the control system to generate corresponding video images for presentation on a display screen, and operate the display screen to simultaneously display video images of the target tissue under broad spectrum light and video images of the target tissue under narrow spectrum excitation light.

12. The method of claim 11, wherein the step of simultaneously displaying video images of the target tissue under broad spectrum light and video images of the target tissue under narrow spectrum excitation light further comprises:
displaying, in rapidly alternating fashion, said video images of the target tissue under broad spectrum light and video images of the target tissue under narrow spectrum excitation light, in the same position in the display screen, so that narrow spectrum excitation light images are superimposed on broad spectrum light images, alternating at an acceptable video frame rate.

13. The method of claim 11, wherein the step of simultaneously displaying video images of the target tissue under broad spectrum light and video images of the target tissue under narrow spectrum excitation light further comprises:
displaying, simultaneously, side-by-side video images of the target tissue under broad spectrum light and video images of the target tissue under narrow spectrum excitation light.

14. A method of visualizing diseased tissue and healthy tissue that may contain a fluorescent agent, said method comprising the steps of:
placing a first video camera in position to obtain video images of the target tissue;
placing a broad spectrum light in position to illuminate the target tissue;
placing a narrow spectrum excitation light in position to illuminate the target tissue, wherein said narrow spectrum excitation light source has a wavelength which will induce fluorescence in a fluorescing agent corresponding to the fluorescence-inducing agents;
operating a control system to alternatingly operate the broad spectrum light and the narrow spectrum excitation light to illuminate the target tissue;
operating the first video camera, through the control system, to obtain video images through the first camera, to obtain, in rapidly alternating fashion, video images of the target tissue under white light and video images of the target tissue under blue light; and
operating the control system to generate corresponding video images for presentation on a display screen, and operate the display screen to simultaneously display video images of the target tissue under broad spectrum light and video images of the target tissue under narrow spectrum excitation light.

15. The method of claim 14, wherein the step of simultaneously displaying video images of the target tissue under broad spectrum light and video images of the target tissue under narrow spectrum excitation light further comprises:
displaying, in rapidly alternating fashion, said video images of the target tissue under broad spectrum light and video images of the target tissue under narrow spectrum excitation light, in the same position in the display screen, so that narrow spectrum excitation light images are superimposed on broad spectrum light images, alternating at an acceptable video frame rate.

16. The method of claim 14, wherein the step of simultaneously displaying video images of the target tissue under broad spectrum light and video images of the target tissue under narrow spectrum excitation light further comprises:
displaying, simultaneously, side-by-side video images of the target tissue under broad spectrum light and video images of the target tissue under narrow spectrum excitation light.
